(19) United States
(12) Patent Application Publication
(10) Pub. No.: US 2019/0232020 A1
(43) Pub. Date: Aug. 1, 2019

(54) ANGIOGRAPHIC CATHETER FOR USE WITH RETROGRADE BLOOD FLOW

(71) Applicant: LifeSAVR Solutions LLC, Minneapolis, MN (US)

(72) Inventors: Jordan M. POLLACK, Minneapolis, MN (US); Michael SEFFREN, Minneapolis, MN (US); Robert F. WILSON, Minneapolis, MN (US)

(21) Appl. No.: 16/180,236

(22) Filed: Nov. 5, 2018

Related U.S. Application Data
(60) Provisional application No. 62/623,288, filed on Jan. 29, 2018.

(51) Int. Cl. A61M 25/01; A61B 6/00; A61M 5/00

(52) U.S. Cl. CPC A61M 25/0105; A61B 6/504; A61M 25/0041; A61B 6/481; A61M 5/007

(57) ABSTRACT

A catheter that is configured to achieve high velocity injections of contrast solution that will not readily wash out in retrograde blood flow. The distal end of the catheter can include a plurality of fluid outlet openings. The fluid outlet openings are spaced circumferentially around the circumference of the catheter and the fluid outlet openings have ejection axes that are angled in a direction toward the distal end so that a velocity component of the ejected contrast solution is against or opposite the retrograde blood flow.

### FIELD

This disclosure relates generally to a catheter for injecting a fluid into a patient. In one specific implementation, the catheter can be an angiographic catheter for injecting a contrast solution into retrograde blood flow within a vessel of a patient during an imaging procedure or an interventional procedure requiring imaging.

### BACKGROUND

Angiographic catheters are well known in the medical field. Angiographic catheters are typically used to inject contrast solution into a vessel of a patient during an endovascular procedure on the patient. Since blood is not visible using imaging technologies such as fluoroscopy (or x-ray), the contrast solution is used to produce a dark image on the screen of the imaging technology being used.

Most standard angiographic catheters are single lumen devices with a fluid outlet opening at the distal end. Some angiographic catheters may have a plurality of small fluid outlet openings located near the distal end of the catheter.

When injecting contrast solution into retrograde (i.e. reverse) blood flow in the vessel with standard angiographic catheters, there is quick washout of the contrast solution. That is, even when used with pressure injectable contrast solution injectors, the contrast solution does not exit the tip of the catheter with enough velocity into the retrograde blood flow and thus the contrast solution washes out quickly from the image resulting in either additional or excessive contrast solution usage, which can be harmful to the patient, or resulting in an unusable image due to lack of contrast solution. In addition, this makes it difficult to accurately image anatomy that is distal to the catheter tip, including, but not limited to, the aortic root during transcatheter aortic valve implantations.

### SUMMARY

A catheter is described that is configured to achieve high velocity injections of contrast solution that will not readily wash out in retrograde blood flow. The catheter can be an angiographic catheter that is configured for injecting a contrast solution into retrograde blood flow within a vessel of a patient during an imaging procedure or any vascular intervention requiring imaging. However, the catheter can be configured for use in other applications where high velocity injections of a solution into a retrograde blood flow are desired.

In one embodiment described herein, the distal end of the catheter includes a plurality of fluid outlet openings in an injection lumen that place a fluid passageway of the injection lumen in fluid communication with an exterior of the injection lumen. The fluid outlet openings are spaced circumferentially around the circumference of the injection lumen, and the fluid outlet openings have ejection axes that are angled in a direction toward the distal end so that a velocity component of the ejected contrast solution is against or opposite the retrograde blood flow.

In another embodiment, the catheter can include a catheter body having a distal end portion with a tip, and a fluid passageway extending through the catheter body to the tip. Axial fluid outlet openings are formed in the tip, where the axial fluid outlet openings extend from the fluid passageway through the tip to place the fluid passageway in fluid communication with an exterior of the catheter body. The axial fluid outlet openings are spaced circumferentially around the circumference of the tip, and the axial fluid outlet openings have ejection axes that are parallel to a longitudinal axis of the catheter body.

In another embodiment, the catheter can include the angled fluid outlet openings in combination with the axial fluid outlet openings.

### DRAWINGS

FIG. 1A illustrates the proximal end portion of a catheter described herein.

FIG. 1B illustrates the distal end portion of the catheter of FIG. 1A.

FIG. 2 is a cross-sectional view of the distal end portion of the catheter of FIG. 1B.

FIG. 3 is a close-up cross-sectional view of the portion of the catheter in the circle 3 in FIG. 2.

FIG. 4 is a close-up cross-sectional view of a distal end portion of a catheter described herein with another embodiment of fluid outlet openings.

FIG. 5 is a close-up cross-sectional view of a distal end portion of a catheter described herein with another embodiment of fluid outlet openings.

FIG. 6 illustrates the distal end portion of another embodiment of a catheter described herein.

FIG. 7A illustrates the distal end portion of another embodiment of a catheter described herein with a double lumen.

FIG. 7B is a cross-sectional view of a portion of the catheter in FIG. 7A.

FIG. 8 illustrates the distal end portion of another embodiment of a catheter described herein.

FIG. 9 is an end view of the distal end portion of FIG. 8.

FIG. 10 is a cross-sectional view of the distal end portion of FIG. 8.

FIG. 11 is a perspective view of another embodiment of a distal end portion of a catheter described herein.

FIG. 12 is a cross-sectional view of the distal end portion of FIG. 11.

FIG. 13 illustrates fluid outlet openings described herein formed through a braided shaft forming the catheter body.

FIG. 14 illustrates another embodiment of an atraumatic tip that can be used on the catheters described herein.

FIG. 15 illustrates an embodiment of a non-straight atraumatic tip that can be used on the catheters described herein.

### DETAILED DESCRIPTION

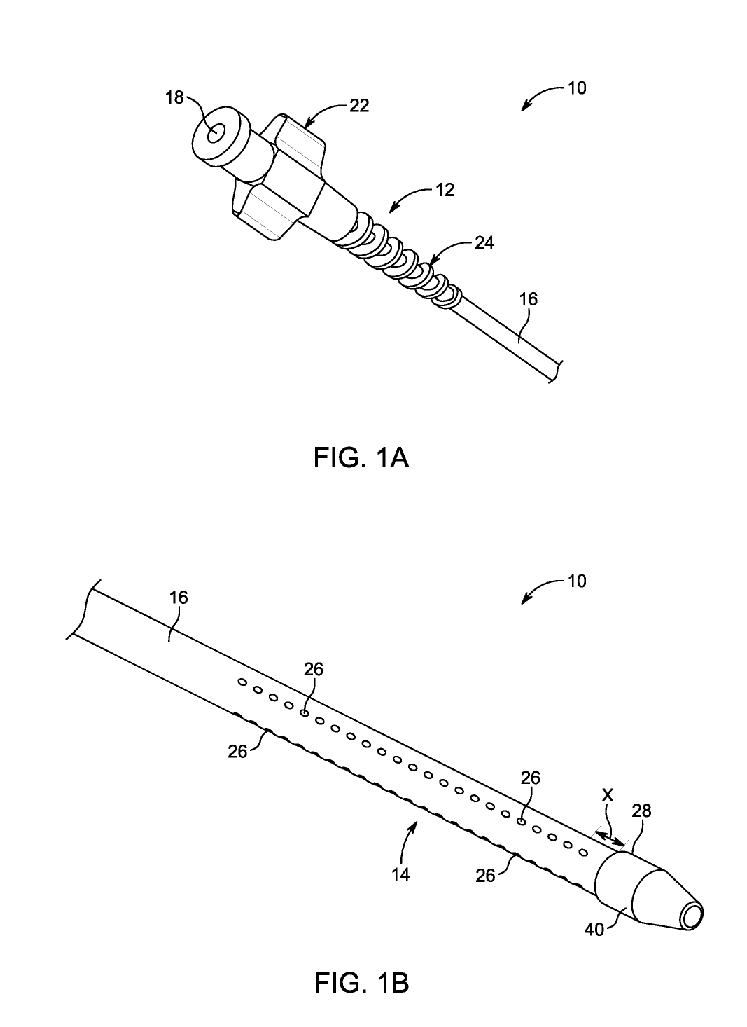

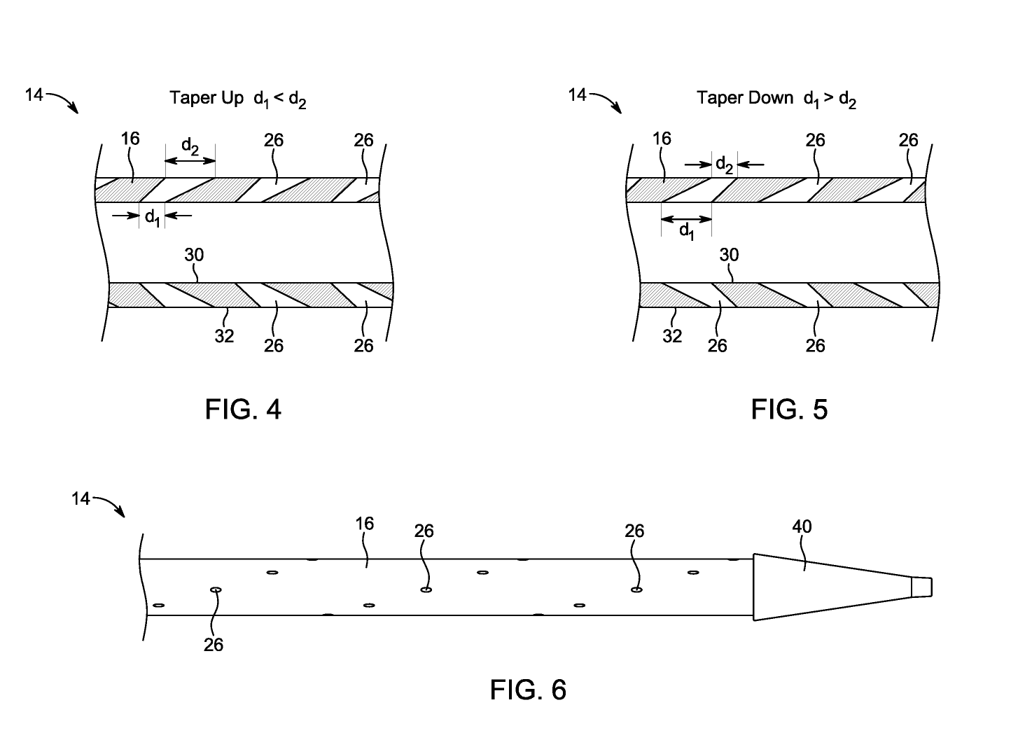

Referring to FIGS. 1A and 1B, an embodiment of a catheter 10 described herein is illustrated. FIG. 1A illustrates a proximal end portion 12 of the catheter 10 while FIG. 1B illustrates a distal end portion 14 of the catheter 10. The catheter 10 has a catheter body 16 formed from any suitable material used to form conventional catheters. A fluid passageway 18 (seen in FIGS. 3 and 4) or lumen extends from the proximal end portion 12 toward and to the distal end portion 14. The catheter 10 is configured to achieve high velocity injections of contrast solution that will not readily wash out in retrograde blood flow. In one embodiment, the catheter 10 can be an angiographic catheter that is configured for injecting a contrast solution into retrograde blood flow within a vessel of a patient during an imaging procedure. However, the catheter 10 can be configured for use in other applications where high velocity injections of a contrast solution into a retrograde blood flow are desired.

Referring to FIG. 1A, the proximal end portion 12 of the catheter 10 includes a luer fitting hub 22 and a strain relief section 24 both of which are well-known in the art. However, other configurations of the proximal end portion 12 are possible. The luer fitting hub 22 can be formed from polycarbonate, HDPE, polypropylene, and other known materials. The strain relief section 24 can be a hard-molded plastic or a soft elastomer like the tip discussed further below.

Referring to FIG. 1B, the distal end portion 14 includes a series of small fluid outlet openings 26 formed through the catheter body 16 that place the fluid passageway 18 in fluid communication with the exterior of the catheter body 16. The fluid outlet openings 26 run in a series along a section of the distal end portion 14 and are arranged circumferentially around the circumference of the distal end portion 14 of the catheter body 16. In FIG. 1B, the fluid outlet openings 26 are illustrated as being arranged in a plurality of linear series, for example 4 linear series, spaced circumferentially around the circumference of the catheter body 16. However, other arrangements of the fluid outlet openings 26 are possible. For example, FIG. 6 illustrates an embodiment of the distal end portion 14 of the catheter where the fluid outlet openings 26 are arranged in a plurality of helical series around the catheter body 16.

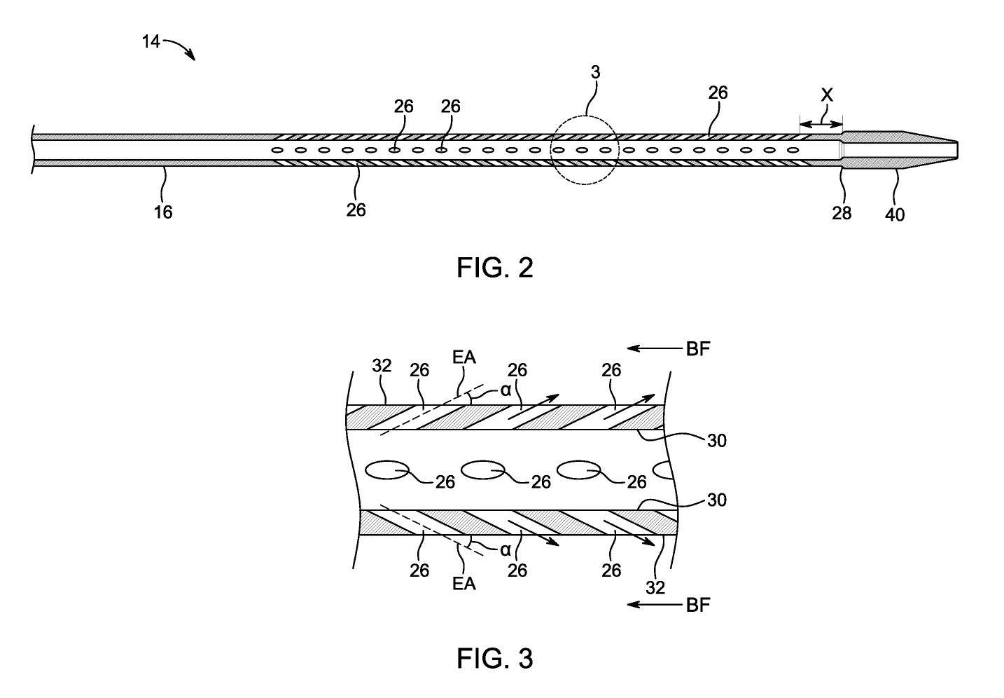

Referring to FIGS. 2 and 3, the fluid outlet opening 26 described herein further have ejection axes (EA) that can be described as being angled in a direction toward a distal end 28 of the distal end portion 14 or of the catheter body 16, or described as being disposed at an acute angle α from the longitudinal axis of the catheter. The angled fluid outlet openings 26 create high velocity fluid flow of fluid (illustrated by arrows in FIG. 3), such as contrast solution, that is ejected through the fluid outlet openings 26 with a velocity component that can be described as being axial or against or opposite retrograde blood flow (BF) within a vessel of a patient. This axial velocity component helps to create longer lasting contrast during an imaging procedure. The acute angle α can be any angle that achieves the desired axial velocity component. The fluid flow exiting the openings 26 also has a radial velocity component that is perpendicular to the axial velocity component. In one embodiment, the acute angle α can be from about 10 degrees to about 75 degrees. In another embodiment, the acute angle α can be from about 30 degrees to about 50 degrees.

The fluid outlet openings 26 can also be described as being holes, slots, apertures, orifices, and the like. The fluid outlet openings 26 can have any suitable shapes (or mix of shapes) as long as the desired axial velocity component can be achieved. Examples of shapes include, but are not limited to, circular, oval, triangular, square, rectangular, pentagonal, hexagonal, septagonal, dodecagonal, star-like, lightning bolt, and the like.

FIGS. 2 and 3 show the fluid outlet openings 26 as having constant dimensions from an inner surface 30 to an outer or exterior surface 32. However, other variations are possible. For example, the fluid outlet openings 26 can be tapered. FIG. 4 illustrates an example where the fluid outlet openings 26 taper outwardly or up so that a size d1 of the openings 26 at the inner surface 30 is less than a size d2 of the openings 26 at the outer surface 32. FIG. 5 illustrates an example where the fluid outlet openings 26 taper inwardly or down so that the size d1 of the openings 26 at the inner surface 30 is greater than the size d2 of the openings 26 at the outer surface 32.

Returning to FIGS. 1B and 2, in one embodiment, the fluid outlet openings 26 can start a distance X or closer from the proximal end of the tip 40 or the boundary between where the tip 40 begins and the catheter body 16. In one non-limiting embodiment, the distance X can be about 0.5 inches or less.

FIGS. 1B and 2 also illustrate a soft tip 40 disposed at the distal end 28 of the distal end portion 14 or the catheter body 16. The soft tip 40 forms a soft, atraumatic tip. The use of a soft, atraumatic tip on a catheter is well known. The soft tip 40 is further designed to allow passage of a guidewire therethrough while preventing the contrast solution from exiting through the soft tip 40 when the contrast solution is injected with high velocities, for example around 1200 psi.

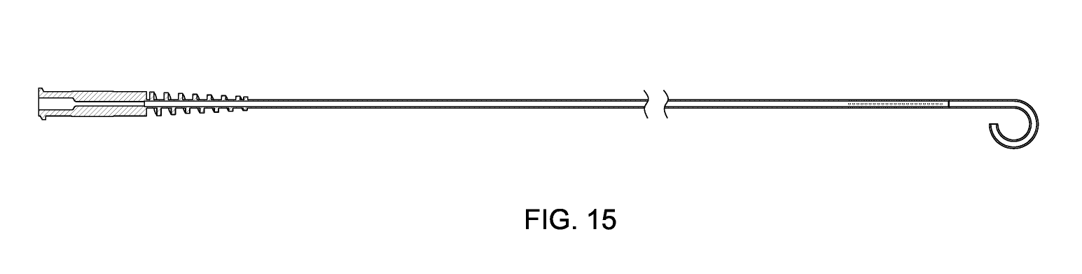

The tip 40 is illustrated in the drawings, such as FIGS. 1B and 2, as being straight from one end thereof to the other end. However, the tip 40 can have other shapes including, but not limited to, a pigtail shape, a hook shape, a helical shape, or other non-straight shape. FIG. 15 illustrates one non-limiting example of the tip 40 having a non-straight shape, in this example a pigtail shape. If a non-straight tip is used, the non-straight tip can maintain its non-straight shape unless a guidewire is inserted through the lumen through the tip thereby straightening the tip.

The tip 40 can be made of any soft elastomeric material(s) suitable for the functions to be performed by the tip 40. Examples of suitable materials include, but are not limited to, soft polymers such as silicone, polyurethane, high-density polyethylene (HDPE), low-density polyethylene (LDPE), Pebax, or other elastomeric material. In one non-limiting example, the tip 40 can have a durometer of anywhere from about Shore 20A to about Shore 70D.

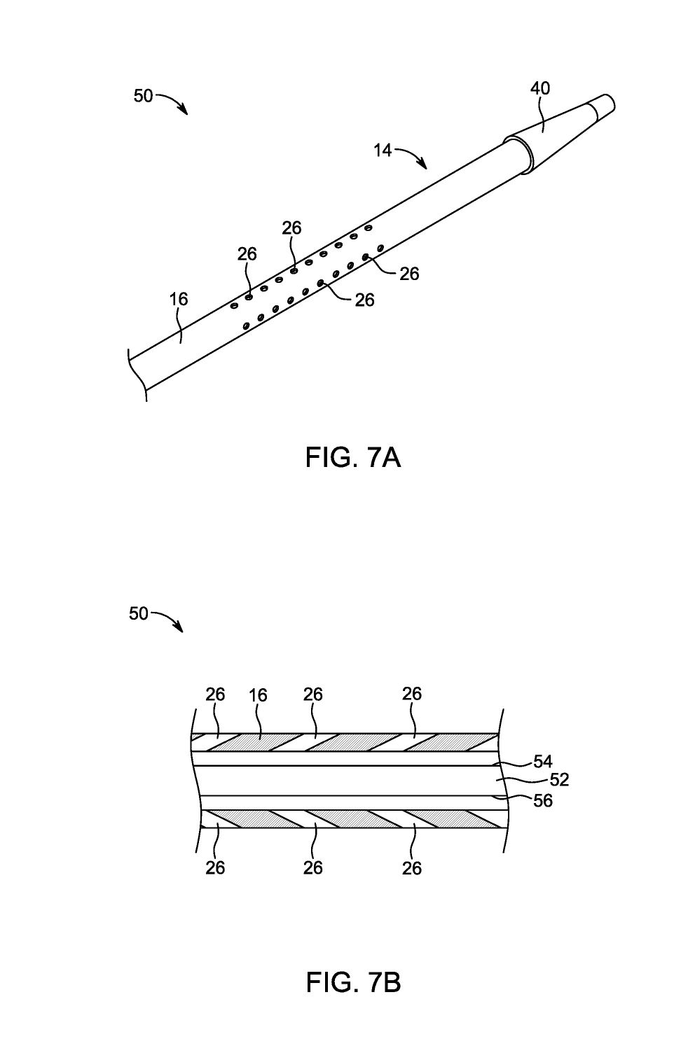

Referring to FIGS. 7A and 7B, another embodiment of a catheter 50 is illustrated. In this embodiment, the catheter 50 includes an inner lumen 52 and an outer lumen 54 (or fluid passageway) for fluid flow. The inner lumen 52 serves as a guidewire lumen through which a guidewire can pass and the outer lumen 54 forms an injection lumen through which the contrast solution passes before being ejected from the fluid outlet openings 26. This allows for controlled flow of the contrast solution without interaction with the guidewire, with the contrast solution being contained in the outer lumen 52. The fluid outlet openings 26 can have a configuration similar to the fluid outlet openings 26 described above for FIGS. 1-6.

Only the distal end portion 14 of the catheter 40 is illustrated in FIGS. 7A and 7B. The proximal end can have a configuration similar to the proximal end 12 in FIG. 1A or have a different configuration. The inner lumen 52 is formed by an inner catheter body 56 disposed within, for example concentrically, the catheter body 16. The outer lumen 54 is formed between the catheter body 16 and the inner catheter body 56. The inner catheter body 56 can be continuous without openings or holes so that the contrast solution in the outer lumen 54 cannot flow into the inner lumen 52.

The catheter 50 further includes the tip 40. The tip 40 can be secured to the inner catheter body 56 or to the catheter body 16. The tip 40 can be made of elastomeric material as described above. However, in one embodiment, the tip 40 can have a hard proximal section (i.e. a larger durometer than the distal section of the tip) to bond to the distal end of the inner catheter body 56 or to the distal end of the catheter body 16. If the tip 40 has a non-straight shape, for example as illustrated in FIG. 15, the tip 40 can be bonded to either the end of the inner catheter body 56, to the end of the catheter body 16, or to the ends of both of the catheter bodies 16, 56. In one embodiment, if a non-straight tip is used, the non-straight tip may have only one lumen for the guide wire, with fluid flow being restricted to the outer lumen only. However, in other embodiments, fluid may also flow through the inner lumen and ultimately through the tip.

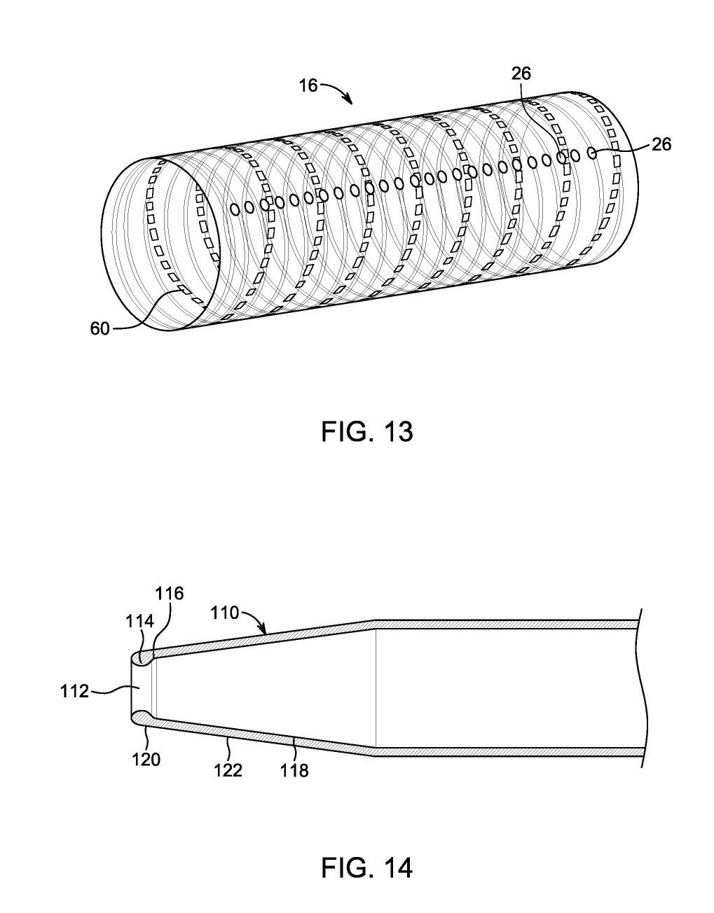

Referring to FIG. 13, the catheter body 16 described herein can have a construction suitable for supporting the high pressure of the contrast solution to be injected. For example, in one non-limiting embodiment, the catheter body 16 can be braided with a strengthening material 60 including but not limited to, stainless steel, titanium, nitinol, and other material to support the high pressure. In addition, the fluid outlet openings 26 can be braided or cut around or circumventing the braiding. In addition, the catheter body 16 and/or the fluid outlet openings 26 can be coated with a hydrophobic coating.

A general method of construction of the catheter 10 is to extrude the catheter body 16 from a standard nylon, polyethylene, PTFE, PFA, Pebax, or others. The catheter body 16 can then be reflowed over PTFE and braiding. The braiding can be stainless steel, titanium, nitinol, or aluminum. The fluid outlet openings 26 can then be laser cut using a laser placed at an angle to create the desired acute angle α. The tip 40 is then over-molded or bonded on the distal end 28. The luer fitting hub 22 and the strain relief 24 are then over-molded or bonded on to the proximal end of the catheter body 16.

In some embodiments, the catheter can include axial fluid outlet openings for the contrast solution either by themselves or in combination with the fluid outlet openings 26. The axial fluid outlet openings would eject the contrast solution into the retrograde blood flow with a velocity component that is substantially entirely axial.

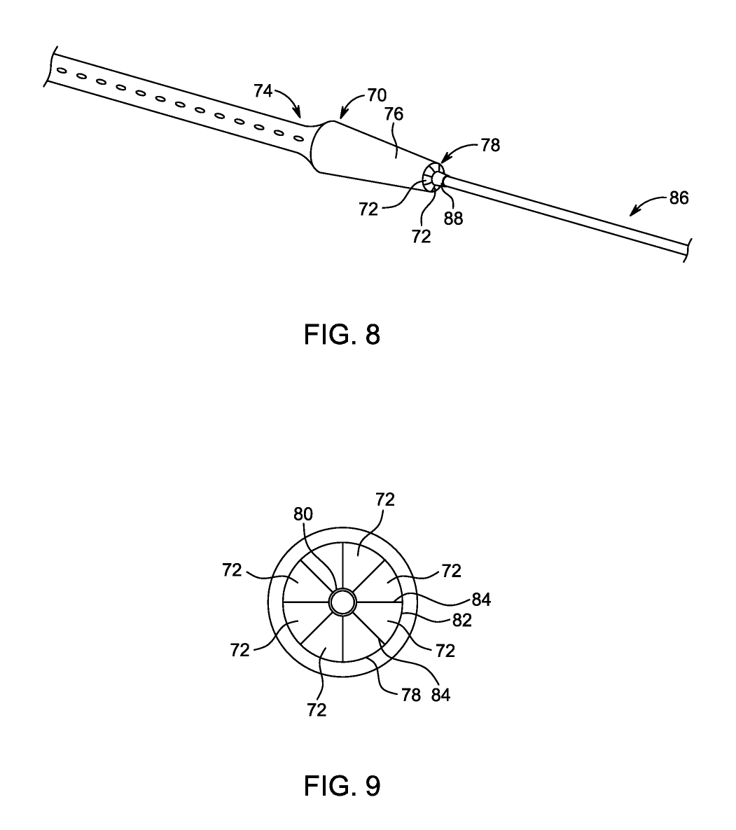

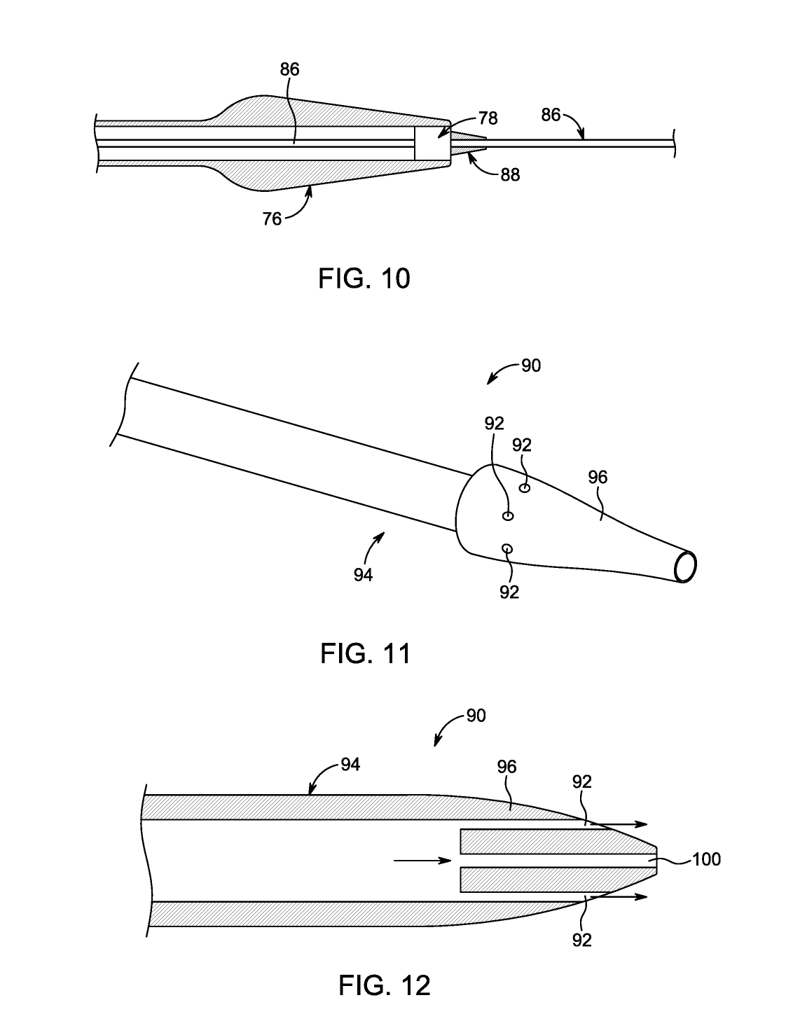

FIGS. 8-10 illustrate an embodiment of a catheter 70 with axial fluid outlet openings 72. FIGS. 8-10 illustrate just a distal end portion 74 of the catheter 70. The proximal end portion can have a configuration similar to the proximal end portion 12 or have a different configuration. In this embodiment, the catheter 70 includes a tip 76 that can have a construction that is similar to the tip 40 or the tip 76 can be non-straight as described above and as illustrated in FIG. 15. A metal or plastic insert 78 that is similar in construction to an apple core device, is placed in the tip 76. The insert 78 includes a center hub 80, an outer wall 82, and a plurality of ribs 84 extending between the center hub 80 and the outer wall 82, with the spaces between the ribs 84 forming the fluid outlet openings 72 that are spaced circumferentially from one another. The center hub 80 allows passage of a guidewire 86, and an optional strain relief 88 can be provided that extends from the center hub 80. The axial fluid outlet openings 72 create straight fluid flow out the tip 76 that is completely (or substantially completely) axial in a direction opposite the retrograde blood flow. In this embodiment, the ejection axes of the axial fluid outlet openings 72 are parallel (or substantially parallel) to the longitudinal axis of the catheter 70. The axial fluid outlet openings 72 may be present in both a straight tip, like the tip 40, or in a non-straight tip that has been straightened by a guidewire.

Referring to FIGS. 11 and 12, another embodiment of a catheter 90 with axial fluid outlet openings 92 in a straight tip 96 is illustrated. FIGS. 11-12 illustrate just a distal end portion 94 of the catheter 90. The proximal end portion can have a configuration similar to the proximal end portion 12 or have a different configuration. In this embodiment, the catheter 90 includes the tip 96 that can have a construction that is similar to the tip 40. In another embodiment, the tip 96 can have a different shape, such as a pigtail shape, a hook shape, a helical shape, or other non-straight shape as illustrated in FIG. 15. A plurality of the axial fluid outlet openings 92 are formed in the tip 96 at circumferentially spaced locations through which contrast solution can be ejected completely (or substantially completely) axial in a direction opposite the retrograde blood flow. In this embodiment, the ejection axes of the axial fluid outlet openings 92 are parallel (or substantially parallel) to the longitudinal axis of the catheter 90. A guidewire (not illustrated) can extend through a central passage or lumen 100 at the center of the tip 96. The axial fluid outlet openings 92 may be present in both a straight tip, like the tip 96, or in a non-straight tip that has been straightened by a guidewire.

FIG. 14 illustrates another embodiment of an atraumatic tip 110 that is usable with any of the catheters described herein. The tip 110 can be used with a catheter having the angled fluid outlet openings, for example as described with respect to FIGS. 1-7. In addition, the tip 110 can be provided with the axial fluid outlet openings, for example as described with respect to FIGS. 8-12. In this embodiment, the tip 110 has an opening 112 for a guidewire, and a rounded lip 114 defining the opening 112. The diameter of the opening 112 is less than the diameter of the guidewire extending therethrough during use. Therefore, there is an interference fit between the opening 112 and the guidewire. In addition, the rounded lip 114 will prevent the opening 112 from blowing open under the pressure of the contrast solution being injected as it will be a softer edge for the contrast solution to impinge against. In this embodiment, the rounded lip 114 is illustrated as being substantially continuously curved from a point 116 within the tip 110 where the lip 114 intersects an angled inner surface 118 of the tip 110 to a point 120 on the exterior of the tip 110 where the lip 114 intersects an angled outer surface 122 of the tip 110.

Any of the catheter embodiments described herein can include a built-in side port and hemostatic valve each of which is known in the art. In addition, any of the catheters described herein can be made steerable (1-axis or 2-axes) in conventional manner to be able to adjust the tip, whether straight or non-straight, location in-vivo. Further, any of the catheters described herein could be configured to have multiple fluid passageways or lumens for multiple fluid injections, for example up to four fluid passageways. Further, any of the catheters described herein can have directional control of the ejected contrast solution where one or more of the fluid outlet openings described herein can be closed off to allow for directionally controlled fluid flow.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A catheter comprising:
a catheter body having a proximal end portion, a distal end portion with a distal end, an exterior surface defining a circumference, and a fluid passageway extending from the proximal end portion to the distal end portion;
a plurality of fluid outlet openings formed in the catheter body adjacent to the distal end portion thereof, the fluid outlet openings extend from the fluid passageway through the exterior surface to place the fluid passageway in fluid communication with an exterior of the catheter body;
the fluid outlet openings are spaced circumferentially around the circumference of the catheter body; and
each one of the fluid outlet openings has an ejection axis that is angled in a direction toward the distal end.

2. The catheter of claim 1, wherein the ejection axis of each one of the fluid outlet openings is disposed at an acute angle from a longitudinal axis of the catheter body.

3. The catheter of claim 2, wherein the acute angle is about 10 degrees to about 75 degrees.

4. The catheter of claim 1, wherein the catheter body has in inner surface; and each one of the fluid outlet openings has:
a constant dimension from the inner surface to the exterior surface; or
a varying dimension where a size of the fluid outlet opening at the inner surface differs from a size of the fluid outlet opening at the exterior surface.

5. The catheter of claim 1, wherein the fluid outlet openings are arranged on the catheter body in a plurality of linear series that are spaced circumferentially around the circumference of the catheter body, or the fluid outlet openings are arranged on the catheter body in a plurality of helical series.

6. The catheter of claim 1, further comprising a tip connected to the distal end, and a plurality of axial fluid outlet openings formed in the tip.

7. The catheter of claim 1, further comprising a tip connected to the distal end, and the tip is straight from one end thereof to an opposite end thereof, or the tip is non-straight from the one end thereof to the opposite end thereof.

8. The catheter of claim 1, wherein the catheter body includes a side port and a hemostatic valve connected to the side port.

9. An angiographic catheter configured for injecting contrast solution into retrograde blood flow in a vessel of a patient, the angiographic catheter comprising:
a catheter body having a proximal end portion, a distal end portion with a distal end, an exterior surface defining a circumference, and a fluid passageway extending from the proximal end portion toward the distal end portion;
a luer fitting hub at the proximal end portion;
a strain relief section adjacent to the luer fitting hub;
a series of fluid outlet openings formed in the catheter body adjacent to the distal end portion thereof, the fluid outlet openings extending from the fluid passageway through the exterior surface to place the fluid passageway in fluid communication with an exterior of the catheter body;
the fluid outlet openings extend along a length of the catheter body in a direction away from the distal end and the fluid outlet openings are spaced circumferentially around the circumference of the catheter body; and
the fluid outlet openings have ejection axes that are angled in a direction toward the distal end whereby contrast solution is ejected through the fluid outlet openings at an acute angle.

10. The angiographic catheter of claim 9, wherein the acute angle is about 10 degrees to about 75 degrees.

11. The angiographic catheter of claim 9, wherein the catheter body has in inner surface; and
each one of the fluid outlet openings has:
a constant dimension from the inner surface to the exterior surface; or
a varying dimension where a size of the fluid outlet opening at the inner surface differs from a size of the fluid outlet opening at the exterior surface.

12. The angiographic catheter of claim 9, wherein the fluid outlet openings are arranged on the catheter body in a plurality of linear series that are spaced circumferentially around the circumference of the catheter body, or the fluid outlet openings are arranged on the catheter body in a plurality of helical series.

13. The angiographic catheter of claim 9, further comprising a tip connected to the distal end, and a plurality of axial fluid outlet openings formed in the tip.

14. The catheter of claim 9, further comprising a tip connected to the distal end, and the tip is straight from one end thereof to an opposite end thereof, or the tip is non-straight from the one end thereof to the opposite end thereof.

15. The catheter of claim 9, wherein the catheter body includes a side port and a hemostatic valve connected to the side port.
